(19) United States
(12) Patent Application Publication
(10) Pub. No.: US 2013/0236529 A1
(43) Pub. Date: Sep. 12, 2013

(54) COMPOSITION AND METHODS TO ENHANCE ANTI-OXIDATION, GUT FLORA AND IMMUNITY IN PETS

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/416,759

(22) Filed: Mar. 9, 2012

(51) Int. Cl. A61K 9/127; A61P 37/02; A61P 1/00; A61K 39/00

(52) U.S. Cl. 424/450; 424/184.1

(57) ABSTRACT

The presented composition and methods are effective to enhance anti-oxidation, gut flora and immune stimulation in pets. The composition includes a combination of Dihydroquercetin and Siberian or Dahurian Larch Arabinogalactan, more specifically, wherein the Larch Arabinogalactan has a molecular weight between about 9.5 kDa and about 37 kDa. The compositions may be in the form of a tablet, chewable tablet, capsule, resin, oil, liquid, liposome, powder, or suppository, and may be further comprised of a pharmaceutically acceptable carrier; or may be incorporated into a bulk feed or a feed supplement.

### FIELD OF INVENTION

This invention and disclosure relates health and nutritional compositions for pets, and more particularly a composition of Dihydroquercetin and Siberian Dahur Larch Arabinogalactan in the form of a tablet, chewable tablet, powder, liquid, resin or oil, and a pre-mixed composition to be added to pet food and methods to enhance anti-oxidation, gut flora and immunity in pets with the administration of the health and nutritional composition.

### BACKGROUND OF INVENTION

The relationship between oxidative stress and human health, particularly in regards to circulatory function and the risk of malignancy is now well accepted science. Harmon, D, 1067 Ann N Y Acad Sci 10-21 (2006). Thousands of published studies implicate free radicals in the development of degenerative disease and accelerated aging. Knight J A. 28(6) Ann Clin Lab Sci. (1998). Huang H. Manton K G. 91100-17 Front Biosci. (2004). Young people naturally produce the antioxidant enzymes super oxide dismutase (SOD), catalase, and glutathione peroxidase that reduce and neutralize the free radicals produced during normal physiological processes. Levels of SOD and other critical antioxidant enzymes decline with age, contributing to the scourge of age-related disease and decline.

Pets, similarly to humans, exhibit significant genetic diversity which affects their overall health and nutritional requirements. Free radicals which form upon exposure to the environment or during normal metabolism can be harmful to cell membranes, proteins and genetic material which can have adverse consequences on the quality of the food and to the body as well. Accordingly, the subject formulations comprise a balanced blend of antioxidants with respect to solubility (fat or watersoluble), stage of rancidity in which they act (oxygen scavengers, free radical termination), and tissues in which they concentrate in the body.

Antioxidants are among the most commonly used supplements to help pets with a variety of medical disorders. Antioxidants are specific vitamins (such as vitamin C and vitamin E) and plant and animal compounds (bioflavonoids such as Dihydroquercetin, grape seed extract, quercetin, coenzyme Q-10, ginkgo biloba) that are responsible for maintaining health. Technically, they do not boost the immune system but rather clean up the products of cell damage, the oxidized chemicals that result. Antioxidants also reduce the levels of various chemicals (histamine, interleukin, tumor necrosis factor) that cause cell damage. Because antioxidants minimize damage to cells, they are useful whenever disease is present and immune support is needed.

Using nutritional supplements to boost the immune system in times of illness is very important for any sick pet. While supplements can be used to support the immune system during any illness, veterinarians are often asked to prescribe something for pets with the following problems: any infectious disease, especially feline respiratory disease, kennel cough, distemper, and parvo; cancer; mange; and any condition in older pets that just “aren't acting quite right.” The use of nutritional supplements for pets is growing in popularity and some pet food contains such supplements.

U.S. Pat. No. 6,312,746, to Paluch, describes a multicomponent food product having improved palatability that may contain antioxidant vitamins such as provitamin A carotenes, vitamin C, vitamin E, and mixtures thereof. U.S. Pat. No. 6,914,071, to Zicker, et al, teaches a method of use of one the antioxidant vitamins Vitamin C, L-carnitine, and alpha-lipoic acid to inhibit the mental deterioration of a companion pet over the age of seven. U.S. Pat. No. 6,974,841 and XX, to Rapisarda, describes an antioxidant health and nutrition dosage for dogs respectfully, comprised of anti-oxidant vitamins, B complex vitamins, bioflavonoids, chelated minerals, digestive enzymes, herbs, nutrients, and essential fatty acids; and U.S. Pat. No. 7,368,481, also to Rapisarda, describes a more specific formulation for cats that includes numerous antioxidant vitamins such as A, C, E, B complex, lutein, L-Cartinine, fatty acids, and digestive enzymes. U.S. Pat. No. 6,946,488, to Hayek, teaches an inflammation reducing cat food that includes including omega-6 and omega-3 fatty acids. U.S. Pat. No. 6,156,355, to Shields, teaches several lengthy and detailed formulations for specific groups of breeds of dogs. U.S. Pat. No. 7,211,280, to Young, describes a method of improving the condition and/or increasing the longevity of elderly cats by administering calcium with vitamins D, E and K, and .beta.-carotene. U.S. Pat. No. 7,129,230, to Inpanbutr, describes a method of treating canine squamous carcinoma with a one of several vitamin D analogs or their stereoisomers.

U.S. Pat. No. 7,399,481, to Sergheraert, teaches a domestic carnivore food composition for the treatment of pigment and quality of fur with animal or plant protein, fatty substances, carbohydrates and at least 5% by weight tyrosine. U.S. Pat. No. 7,261,888, to Decuypere, et al, teaches a feed composition for use as an anti-microbial and prevention of digestive upsets comprising triglycerides, medium chain fatty acids, and exogenous active lipolytic enzyme. U.S. Pat. No. 7,258,879, to Hodge, describes a packaged treat-sized pet food product to reduce flatulence odor utilizing vegetable charcoal and Yucca Shidigera or Elata extract. U.S. Pat. No. 7,244,463, to Wadsworth, teaches the use of garcinia mangostana L. in a pet food product. U.S. Pat. No. 6,669,975, to Abene, teaches a customized pet food product of pre-made kibble and mixtures of functional ingredients green lipped mussel extract, borage oil, sunflower oil, flax seed oil, flax seeds, fish oil, L-carnatine, conjugated linoleic acid, fructooligosaccharide, vitamin E oil, ground chicory, vitamin C, SAM-E, Gamma linoenic acid, evening primerose oil, soy isoflavones, creatine, herbs, mannan oligosaccharides, and antioxidants. U.S. Pat. No. 7,332,188, to Axelrod, discloses an animal chew toy fortified with fermented soya.

None of these teachings describe, nor is there available on the market, a feed or health and nutrition composition for pets that provides the unique properties of dihydroquercetin and Siberian or Dahurian Larch Arabinogalactan.

### Dihydroquercetin

Dihydroquercetin is a bioflavonoid. Flavonoid natural products display a wide range of biochemical and pharmacological properties, with one of the most thoroughly characterized effects being chemopreventive activity, measured by the chemopreventive index as a marker for the screening of potential agents. Dihydroquercetin, also known as taxifolin, exhibits high detoxification ability but lower cytotoxicity in cells, representative of a high chemo-preventive index. It has been shown to modulate the expression of several genes, including those coding for detoxification enzymes, cell cycle regulatory proteins, growth factors, and DNA repair proteins. A recent microarray results show that the phase II detoxification enzymes, NQO1 and GSTM1, are upregulated, while the phase I detoxification enzyme, CYP2E1, is down regulated in the presence of taxifolin. Lee, S B, et al, 30(6) Biol Pharm Bull 1074-1079 (2007). Measurement of cytotoxicity to human lung embryonic fibroblasts (TIG-1) and umbilical vein endothelial (HUVE) cells, was examined and Dihydroquercetin shown to be the least cytotoxic of the ten (10) tested flavonoids, with a 50% lethal concentration of >300 micromoles in TIG-1 and >200 micromoles in HUVE cells. Matsuo, M, et al., 28(2) Biol Pharm Bull 253-9 (2005).

Dihydroquercetin is an excellent synergist with vitamins and co-factor to enzyme antioxidant systems in catalyzing the donation of electrons, with a high Oxygen Radical Absorbance Capacity (ORAC) value that inhibits the donation of hydrogen by lipid peroxides, and its enzyme forms are metal chelator. Dihydroquercetin can quickly metabolize into enzyme forms in blood and works as the catalyst for human antioxidant system. Dihydroquercetin is a far more powerful antioxidant than vitamins A, C and E, as demonstrated by its USDA ranking of the ORAC and additional studies.

Commonly peroxide radical relates to oxidative damage of fatty acids and other lipids, and demonstrates why radicals such as both peroxide and hydroxyl radicals can cause so much more damage than one might have expected. According to the chemical reactions involved, major antioxidant capacity assays are essentially divided into two categories: (1) hydrogen atom transfer (HAT) reaction based assays and (2) single electron transfer (ET) reaction based assays.

The majority of HAT-based assays apply a competitive reaction scheme, in which antioxidant and substrate compete for thermally generated peroxyl radicals through the decomposition of azo compounds. ORAC is an assay for the inhibition of induced low-density lipoprotein oxidation and oxygen radical absorbance capacity. The ORAC hydro tests the capacity dihydroquercetin generated hydrogen atom transfer (HAT) to compete for thermally generated peroxyl radicals and hydroxyl radicals. For the latter it is necessary to use the ORAC assay test. Unlike electron transfer based assays that measure an antioxidant's reducing capacity, and the hat-based assays measure the hydrogen atom donating capacity. The hydrogen atom transfer is a key step in the radical chain reaction. Therefore, the hat-based assays are more relevant to the radical chain-breaking antioxidant capacity. Among all the hat-based assays, ORAC adopted an AUC (area under curve) technique to quantify antioxidant capacity. The advantage of the auc approach is that it applies equally well for both antioxidants that exhibit distinct lag phases and those samples that have no lag phases. This approach unifies the lag time method and initial rate method, and it is particularly useful for food samples, which often contain multiple ingredients and have complex reaction kinetics. Therefore, the ORAC assay has been broadly applied in academics and the food and supplement industry as the method of choice to quantify antioxidant capacity. In fact, an antioxidant database has been generated applying the orac assay in combination with the total phenols assay. The orachydro value of our dihydroquercetin has been measured at least is 300 micromole of trolox equivalent per 420 mg capsule or 800 micromole trolox equivalent per gram.

Dihydroquercetin is a powerful antioxidant and is one substance in foods that significantly decreases the adverse effects of radical oxygen species, and reactive nitrogen species. Valko, M et al, 39(1) Int J Biochem Cell Biol. 44-84 (2007); Kostyuk V A, Potapovich A I, 355(1) Arch Biochem Biophys 43-8 (1998); Soliman K F & Mazzio E A, 218(4) Proc Soc Exp Biol Med 390-7 (1998). It was discovered as an essential molecular intermediate in flavonoid biosynthetic pathways responsible for quercetin, oligo proanthocyanidins (OPC), and catechin formation and its superiority as an anti-oxidant has been demonstrated repeatedly. Tiukavkina N A, Rulenko I A, Kolesnik I A, 6 Vopr Pitan. 12-5 (1997); Teselkin, I O, et al, 41(3) Biofizika. 620-4 (1996).

Dihydroquercetin protects cell membranes from free radical damage, improves the activity of capillaries and supports the recovery of blood micro-circulation throughout the body. Oxidative stress is reduced at the cellular level, as shown, for instance in cultured renal cells. Areias, F M, et al, 62(1) Biochem Pharmacol. 111-8 (2001). Due to Dihydroquercetin's permeability into human cells including blood cells, dihydroquercetin has demonstrated reliable protection at the cellular level from a variety of reactive free radical species.

The plasma levels of Dihydroquercetin remain higher longer than ordinary quercetin, as Dihydroquercetin is not broken down by the body's enzymes to less active compounds in quantities near the extent that quercetin has been. In addition, it is 400 times less mutagenic than quercetin; quercetin has a minimum mutagenic dose of 4 nml to 1626 nmol for Dihydroquercetin. Jurado, J., 25 et al, 6(4) Mutagenesis 289-95 (1991).

Dihydroquercetin has been shown to inhibit the production of luminol-dependent chemiluminescence (CLlum) by activated human neutrophils, 'T Hart Chem Biol Interact. 1990;73(2-3):323-35, and to inhibit their adhesion during inflammation. Wang, Y H, et al, 67(12) Biochem Pharmacol 2251-62 (2004). Dihydroquercetin is reported to protect phagocytic cells from silica induced injury. Kostyuk V A, Potapovich A I. 355(1) Arch Biochem Biophys 43-8 (1998). Dihydroquercetin was shown by active as an inhibitor of histamine release by mast cells. Bronner C, Landry Y., 16(3) Agents Actions 147-51 (1985).

The partition coefficients of both catechin and taxifolin in a biomimetic system (micelles) were determined, since these properties may also contribute to the antioxidant behavior of this type of compound. The log P values determined depend on the electrostatic interactions of the compounds with the differently charged micelles (the highest values were obtained for zwitterionic and cationic micelles). The pro-oxidant behavior of the compounds was assessed through the oxidation of 2′-deoxyguanosine, induced by a Fenton reaction, catalyzed by copper. The data obtained reveal that the flavonoids under study did not present pro-oxidant activity, in this particular system. Teixeiria, S, 39(8) Free Radic Biol Med 1099-108 (2005).

Cardiovascular function is also supported by flavonoids particularly dihydroquercetin. It has been shown to inhibit lipoxygenase, the enzyme that oxidizes lipids in fatty acids. Wheeler, E L, and Berry, D L, 7(1) Carcinogenesis 33-6 (1986). In combination with Vitamin C, Dihydroquercetin increases erythrocyte deformability and decreases their aggregation. Plotnikov, M B, et al, 104(12) Zh Nevrol. Psikhiatr Im S S Korsakova 33-7 (2004). Improved resistance of microsomes to lipid peroxidation has also been demonstrated for dihydroquercetin. Kravchenko L V, Morozov S V, Tutel'yan V A, 136(6) Bull Exp Biol 572-5 (2003).

It is recognized that Siberian or Dahurian Larch, latter is known colloquially as the mongolian larch tree, are both excellent sources for market volumes of Dihydroquercetin.

### Arabinogalactan

Many herbs with well established immune-enhancing properties, such as Echinacea purpurea, Baptisia tinctoria, Thuja occidentalis, Angelica acutiloba and Curcuma longa also contain significant amounts of AG. Polysaccharides are often found in the medicinal herbs used for immune enhancement, including Echinacea and Astragalus. Arabinogalactans are a class of long, densely branched polysaccharides with molecular weights ranging from 6,000 to 120,000 and more that are found in the cell walls of a wide variety of edible and non-edible woody plants. Arabinogalactan is a fine, dry, off-white powder with a mildly sweet taste that mixes well with liquids. This safe and effective phytochemical is FDA approved for use as a dietary fiber, even at large doses, and as a food additive. 5(5) Altern Med Rev 463-6 (2000) (no authors listed). The only reported side effect is occasional bloating and flatulence in a small percentage of people who take it.

Arabinogalactan powder enhances the immune system by increasing the number of immune cells present in the bloodstream, enhancing natural killer cell and macrophage activation, and limiting unhealthy cell replication. Arabinogalactan powder supports the immune system and colon health by moderating acidic build-up in the intestinal tract. The fiber is non-allergenic and does not stimulate an insulin response.

Arabinogalactans are most abundant in the larch tree (Larix spp.) Larch Arabinogalactan can be extracted from either the Western larch (Larix occidentalls), or Siberian or Dahurian larch (Larix Sibirica or Larix gmelinni), latter is known colloquially as the Mongolian larch. The Western larch is most commonly used. Because of their potent biological activity and immune-enhancing properties, this unique dietary fiber is receiving increased attention as a clinically useful nutraceutical agent.

### Gut Flora

Larch is used medicinally for the effects of these polysaccharides on the intestines and immune system. Larch Arabinogalactan is a non-digestible soluble dietary fiber that resists breakdown by enzymes and enters the large bowel intact where it is fermented by colonic bacteria.

Many edible and inedible plants are rich sources of Arabinogalactan, including leek seeds, carrots, radishes, black gram beans, pears, maize, wheat, red wine, Italian ryegrass, tomatoes, ragweed, sorghum, bamboo grass and coconut meat and milk.

Siberian or Dahurian Larch Arabinogalactan is believed to act as a prebiotic; it stimulates the colonic growth of such bacteria as bifid bacteria and lactobacilli that confer certain health benefits. Ingestion of Larch Arabinogalactan has a significant effect on enhancing beneficial gut microflora, specifically increasing anaerobes such as Lactobacillus. Robinson R R, Feirtag J, Slavin J L, 20(4) J Am Coll Nutr 279-85 (2001).

Ninety percent of all chronic disease is caused by an unhealthy intestinal tract. In a study of several forms of arabinogalactan (AG) in dogs, (Cohasset, Minn.); raw AG (AG100), Laraceutical (AG1000) and Fiberaid (AG3000) at 1.65 g/day and 0.55 g/day, low dose AG1000 and high dose AG3000 resulted in a trend to higher concentrations of fecal lactobacilli (P=0.04) and higher concentrations of fecal bifidobacteria (P 0.16) compared with control dogs. Dogs fed low dose AG3000 tended (P=0.10) to have a lower concentration of fecal Clostridium perfringens compared with control dogs. Greishop, 132 J Nutr 478-482 (2002). Arabinogalactan powder can limit the progress of a disease by stimulating immune cell activation.

Short chain fatty acids, primarily acetate, propionate, and butyrate, are produced in the colon by fermentation of dietary carbohydrates, particularly from degradation-resistant starches and dietary fiber, play an important role in intestinal health. These acids are the principal energy source for the colonic epithelial cells. The non-absorbed fiber of Arabinogalactan is eagerly fermented by the distal gut microflora, resulting in an elevated production of short-chain fatty acids, primarily butyrate, and, to a lesser extent, propionate. Ammonia is produced as a by-product in the colon by bacterial fermentation of protein and other nitrogen-containing substances. Research indicates that ammonia levels as low as 5 mmol/L can have detrimental effects on epithelial cells that line the colon. The toxicity of ammonia toward colonic epithelial cells can lead to cell destruction and increased turnover of these cells.

Many clinicians use prebiotics as a supplemental support for intestinal conditions including diverticulosis, leaky-gut, irritable bowel syndrome as well as inflammatory bowel diseases such as Crohn's disease and ulcerative colitis. Studies have shown that Larch Arabinogalactan consumption reduces intestinal ammonia generation. Robinson R R, Feirtag J, Slavin J L, 20(4) J Am Coll Nutr 279-85 (2001). Since even low ammonia levels can have damaging effects on intestinal colonic cells, Larch Arabinogalactan may support patients who are unable to detoxify ammonia. When exposed to bifidobacteria, as those found in the healthy gut, fermentation of arabinogalactan is low contrary to the oligosaccharides derived from it. Degnan, B A, McFarlane, G T, 1(2) Anaerobe 103-12 (1995). Oligosaccharides are also a source of dietary fiber. Gray, J., Dietary Fiber, ILS1 Europe Concise Monograph Series (2006).

### Immune Modulation

Arabinogalactan supports healthy immune system functioning. Schepetkin, I A, 5(13-14) Int Immunopharmacol 1783-99. (2005). Larch Arabinogalactan, in particular, increases the levels of [25] beneficial immune cells such as white blood cells without stimulating pro-inflammatory immune cell production or activation. Kelly, G S, 4(2) Ahern Med Rev 96-103 (1999). Larch Arabinogalactan decreases the production of lymphoid cells in bone marrow. Currier, N L, 10(2-3) Phytomedicine: 145-53 (2003). It is highly effective in promoting in vitro and in vivo macrophage proliferation and exhibits anti-inflammatory, anti-complement, and anti-allergic effects. Kim L S, Waters R F, Burkholder P M., 7(2) Altern Med Rev 138-49 (2002).

Several studies have shown that Larch Arabinogalactan have anti-cancer activity in animals. In a 1987 study reported in the Journal of Cancer Research and Clinical Oncology, researchers tested their theory that organ-specific lectins (which represent a type of protein) help determine where malignant cells will metastasize in animals. Injections of arabinogalactans were shown to effectively block these lectins in mice livers and therefore prevent the spread of this mouse cancer. Beuth, J, 10 113(1) J Cancer Res Clin Oncol 51-5 (1987).

This study was replicated successfully by researchers in Cologne, Germany, and published in 1988. Beuth, J, 6(2) Clin Exp Metastasis 115-20 (1988). A similar study in Sweden in 1991 found that pre-treatment with arabinogalactans reduced the number of liver metastatis and lengthened survival times in laboratory animals. Hagmar B, Ryd W, Skomedal H, 11(6) Invasion Metastasis 348-55 (1991).

A review of Larch Arabinogalactan research published in the Alternative Medicine Review in 1999 reported that the substance can stimulate natural-killer-cell activity and enhance other aspects of immune function as well. Kelly, G S, 4(2) Altern Med Rev 96-103 (1999).

Larch Arabinogalactan enhances immune response in part due to its ability to stimulate “natural killer” (NK) cell cytotoxicity, a functional marker for health. Reports in the medical literature link decreased NK cell activity to a variety of chronic diseases including chronic fatigue syndrome, viral hepatitis, HIV/AIDS, and autoimmune diseases such as multiple sclerosis. Modified citrus pectin has the same anti-metastatic mechanism of action in animals as Larch Arabinogalactan, but does not provide the immune-modulating effects.

In a well-controlled study, Larch Arabinogalactan induced an increased release of interferon gamma (IFN gamma), tumor necrosis factor alpha, interleukin-1 beta (IL-1 beta) and interleukin-6 (IL-6). This resulted in activating two powerful cells of the immune system: macrophages and NK cells. It was found that the IFN gamma was most responsible for the observed enhancement of NK cytotoxicity. Hauer J, Anderer F A., 36(4) Cancer Immunol Immunother 237-44 (1993).

Other reports indicate Larch Arabinogalactan has also been shown to support a decrease in the frequency and severity of pediatric otitis media caused by gram negative rods, especially Escherichia coli and Klebsiella. D'Adamo P., 6 J Naturopathic Med 33-37 (1996). Larch. Arabinogalactan has been isolated and characterized as a low molecular weight, 9 kDaltons Arabinogalactan fragment and a purified Arabinogalactan, 37 kDa, from Larix occidentalis that is composed of repeating units of similar molecular weight and composition. A 9 kDa Arabinogalactan may be obtained in high yield from 37 kDa Arabinogalactan either by autoclaving at 121 degrees C. or by exposure to alkaline solution in the presence of sodium borohydride. The weight average molecular weight of 37 kDa Arabinogalactan was determined to be 37 and 38 kDa by intensity light scattering and sedimentation equilibrium, respectively. The weight average molecular weight of 9 kDa Arabinogalactan was determined to be 6 and 9.5 kDa by intensity light scattering and sedimentation equilibrium, respectively. MALDI-TOF mass spectrometry yielded a molecular weight at the peak of the distribution of 8.3 kDa for Arabinogalactans (9 kDa). Both Arabinogalactans (37 kDa) and Arabinogalactans (9 kDa) exhibited narrow molecular-weight distributions (Mw/Mn approximately 1.2). Arabinogalactans 25 (37 kDa) and Arabinogalactans (9 kDa) exhibit nearly identical 13C-NMR spectra, monosaccharide composition, and sugar linkages. It is proposed that Arabinogalactans (37 kDa) is composed of covalently bound subunits of Arabinogalactans (9 kDa). Arabinogalactans (37 kDa) and Arabinogalactans (9 kDa) bind isolated hepatocyte glycoprotein receptor equally well. As a result Arabinogalactans (9 kDa) is a candidate for use in hepatocyte directed drug delivery and may be more desirable for such use than is Arabinogalactans (37 kDa). Larch Arabinogalactan in powder form is typically dosed in teaspoons or tablespoons at a concentration of approximately 3 grams per teaspoon.

Because of its mild taste and excellent solubility in water or juice, it is easy to mix with homemade pet food, such as yogurt. This can be done regularly or when a pet needs immune stimulation and protection of the gut microflora on an acute basis.

### SUMMARY OF THE INVENTION

This inventor has discovered that the combination of the potent antioxidant, dihydroquercetin, with the powerful immune modulator Larch Arabinogalactan, particularly Siberian or Dahurian Larch of low molecular weight, will maintain and improve the health of a pet's immune system. While this is usually provided as part of pet's regular diet and supplement regiment, it can also be used as an effective boost of anti-oxidation, gut flora, and the immune system under adverse circumstances.

For instance, there is a viral epidemic, such as parvo, an extra dose of the dihydroquercetin and Siberian or Dahurian Larch Arabinogalactan can be give to the pets to boost immunity and enhance the activity of the gut flora. The composition may be administered as a capsule, tablet, chewable tablet, liquid, resin, oil, powder, liposome, suppository or pre-mix that is sold as part of a complete feed or added to homemade or manufactured pet food and may be further comprised of a pharmaceutically acceptable carrier.

The Siberian or Dahurian Larch is found exclusively in the East Siberia and Far East of Russia, and dihydroquercetin is isolated from its bark and butt logs. Both ingredients are available from FLAVITPURE Inc, USA. Dihydroquercetin is considered to be a dietary antioxidant and is one substance in foods that significantly decreases the adverse effects of reactive oxygen species. It was discovered as an essential molecular intermediate in flavonoid biosynthetically pathways responsible for quercetin, oligo proanthocyanidins and catechins formation.

The health and nutritional composition provides for the promotion of beneficial bacteria while reducing the pathogenic bacteria in the digestive tract of animals; endogenous antioxidant defense and catalytic action for internal antioxidant re-circulation; synergy for essential C, E, A vitamins; enhancement of short chain fatty acids, all of which contribute to a healthier animal. The dihydroquercetin breaks the chain of oxidation in peroxide reactions and enhances the bodies internal enzyme antioxidant system and vitamin re-circulation.

When added to feed, the composition improves feed efficiency and weight gain, lower feed costs per animal, reduction or elimination of the occurrence of scours, and less reliance on antibiotics. It is also an effective feed additive that can be easily incorporated into a homemade or existing formula.

### DETAILED DESCRIPTION OF INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In the Summary above, the Description of the Invention, and the Claims and Abstract below, reference may be made to particular features (including method steps) of the invention. It is to be understood that this disclosure includes possible combinations of such particular features. For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

The term “comprises” and grammatical equivalents thereof are used herein to mean that other components, ingredients, steps etc. are optionally present. For example, an article “comprising” (or “which comprises”) components A, B and C can consist of (i.e. contain only) components A, B and C, or can contain not only components A, B and C but also one or more other components.

The term “at least” followed by a number or the indefinite article “a” (meaning “one”) is used herein to denote the start of a range beginning with that number (which may be a range having an upper limit or no upper limit, depending on the variable being defined).

This inventor has discovered a health and nutrition composition effective to enhance anti-oxidation, 15 gut flora and immune stimulation in pets, comprising Dihydroquercetin and Siberian or Dahurian Larch Arabinogalactan. Specifically, the Larch Arabinogalactan has a molecular weight between about 9.5 kDa and about 37 kDa and the dosage may be in a form of a tablet, chewable tablet, capsule, resin, oil, liquid, liposome powder, or suppository, and may be further comprised of a pharmaceutically acceptable carrier.

In a specific embodiment, the Dihydroquercetin is present in an amount of about 3 percent by weight to an amount of about 30 percent by weight, more specifically about 5 percent by weight to an amount of about 20 percent by weight and most specifically in amounts of either 5 percent by weight or 10 percent by weight. In a specific embodiment, the composition weighs about 3 grams per tablespoon.

Also provided are methods of enhancing anti-oxidation, gut flora and immune stimulation to a pet, particularly a dog, by the administration of the health and nutrition composition. The 5 percent Dihydroquercetin composition may be administered in an amount of about 0.6 grams (0.2 tablespoons) to a dog weighing less than about 10 kg in weight. The 10 percent Dihydroquercetin composition also weighs about 3 grams per tablespoon and may be administered in an amount of about 0.9 grams (0.3 tablespoons) to a dog between about 10 kg and about 50 lbs in weight, and 1.5 grams (0.5 tablespoons) to a dog between about 50 lbs and about 100 lbs in weight, and 2.4 grams (0.8 tablespoons) to a dog over 100 lbs in weight. The method of enhancing anti-oxidation, gut flora and immune stimulation to a pet, may also be employed for a cat, specifically using the 5 percent Dihydroquercetin composition in an amount of 0.1 tablespoons.

In another embodiment of the invention, the health and nutrition composition is pre-mixed before being mixed with and becoming part of a bulk feed for canines comprising meat, grain, fiber, and herbs and spices for natural flavor; and a vitamin mixture comprising tocopherol, Vitamin C, carotenoid, organic minerals, and omega fatty acids. In this embodiment, the premixed Dihydroquercetin and Larch Arabinogalactan is present in an amount of about 0.5 percent by weight to about 5 percent by weight of complete feed, in which meat is present in an amount of about 10 percent by weight to about 50 percent by weight, grain is present in an amount of about 10 percent by weight to about 80 percent by weight, fiber is present in an amount of about 0.1 percent by weight to about 20 percent by weight, herbs and spices are present in an amount of about 0.01 percent by weight to about 0.5 percent by weight; and tocopherol is present in an amount of about 0.01 percent by weight to about 1 percent by weight, Vitamin C is present in an amount of about 0.1% to about 5% by weight, carotenoid is present in an amount of about 0.2 to about 1% by weight, organic minerals are present in an amount of about 0.01 percent by weight to about 0.5 percent by weight, and omega fatty acids are present in an amount of 1 percent by weight to about 5 percent by weight.

In a more specific embodiment, the premixed Dihydroquercetin and Larch Arabinogalactan is present in amount of about 1 percent by weight to about 2 percent by weight of the feed, meat is present in an amount of about 20 percent by weight to about 25 percent by weight, grain is present in an amount of about 25 percent by weight to about 45 percent by weight, fiber is present in an amount of about 0.5 percent by weight to about 4 percent by weight, herbs and spices are present in an amount of about 0.01 percent by weight to about 0.2 percent by weight; mixed tocopherols are 5 present in an amount of about 0.025 percent by weight to about 0.05 percent by weight, said Vitamin C is present in an amount of about 0.2% by weight, carotenoid is present in an amount of about 0.54% by weight, organic minerals are present in an amount of about 0.01 percent by weight to about 0.02 percent by weight, omega fatty acids are present in an amount of 2 percent by weight. The meat may be cow, goat, poultry, and fish and the omega fatty acids may be one or more omega-3 fatty 10 acids, omega-6 fatty acids, and omega-9 fatty acids and may be present in a ratio between about 5:1 to about 10:1 omega-6 to omega-3 fatty acids and said omega-9 fatty acids are present in an amount of about 4 mg. The carotenoid is most specifically beta-carotene or lutein.

Optionally, the health and nutrition composition pre-mixed and mixed with bulk feed may further comprise one or more of magnesium, iron, manganese, zinc, copper, iodine, selenium, Vitamin A, mixed tocopherols, Vitamin D-3, thiamine, riboflavin, niacin, D-pantothenic acid, pyridoxine, biotin, folic acid, choline, vitamin B-12. Most specifically, these nutrient are present in amounts sufficient to provide the following per kilogram of dietary supplement and feed composition: 600 mg magnesium; 124 mg iron; 58.4 mg manganese; 233 mg zinc; 42.2 mg copper; 2.6 mg iodine; and 20 0.34 mg selenium; 27,900 IU vitamin A; 139.1 IU vitamin E; 1,826 IU vitamin D-3; 17.7 mg thiamine; 41.0 mg riboflavin; 69.5 mg niacin; 35.3 mg D-pantothenic acid; 10.0 mg pyridoxine; 0.57 mg biotin; 1.93 mg folic acid; 2416 mg choline; and 0.28 mg vitamin B-12.

Also provided are methods for use of the health and nutrition composition pre-mixed and mixed with bulk feed to enhance anti-oxidation, gut flora, and immune stimulation in a pet. The health and nutrition composition, with the bulk feed, may be administered in the following amounts: about 20 gm to about 70 gm to said dog if said dog weighs 5 kg or less; about 70 gm to about 110 gm to said dog if said dog weighs about 5 kg to about 10 kg; and about 10 gm per kg to said dog if said dogs weighs over about 10 kg.

In another embodiment, a health and nutrition composition effective to enhance anti-oxidation, gut flora and immune stimulation is further comprised of protein, crude fat, crude fiber, moisture, calcium, sodium, chloride, and magnesium and provided in a form suitable to be added to homemade or marketed pet food for immediate enhancement of anti-oxidation, gut flora, and immune stimulation. In this embodiment, the Dihyroquercetin is premixed with the Siberian or Dahurian Larch Arabinogalactan before the protein, crude fat, crude fiber, moisture, calcium, sodium, chloride, and magnesium are added. Specifically, the Larch Arabinogalactan has a molecular weight between about 9.5 kDa and about 37 kDa; the premixed Dihydroquercetin and Larch Arabinogalactan is present in an amount ranging from about 5% to about 95% by weight, protein is present in an amount ranging from about 5% by weight to about 95% by weight, crude fat is present in an of at least 0.01% by weight, crude fiber is present in an amount up to 10% by weight, moisture is present in an amount up to 10% by weight, calcium is present in an amount no less than 0.02% by weight, sodium is present an amount ranging from about 0.02% by weight to about 0.1% by weight, chloride is present in an amount ranging from 0.005% by weight to about 0.02% by weight, and magnesium in an amount ranging from 0.05% by weight to about 0.2% by weight. In its most specific embodiment, the premixed Dihydroquercetin and Larch Arabinogalactan is present in an amount of about 10% by weight, protein is present in an amount of about 80% by weight; crude fat is present in an amount of at least 0.5% by weight, crude fiber is present in an amount up to 3% by weight, moisture is present in an amount up to 5% by weight, calcium is present in an amount not less than 0.05% by weight, sodium is present an amount of about 0.05% by weight, chloride is present in an of about 0.01% by weight, and magnesium in an amount of about 0.1% by weight. The health and nutrition composition may be provided in packages, most specifically about 10 gm packages, to be mixed with homemade or marketed pet food for immediate enhancement of antioxidation, gut flora and immune stimulation. In specific embodiments the Dihydroquercetin is present in an amount of about 0.5% by weight and about 1% by weight.

Also provided are methods of providing immediate enhancement of antioxidation, gut flora, and immune stimulation of a pet, comprising the steps of mixing the health and nutrition composition into a homemade or marketed feed, and providing said mixed composition and feed as one or more meals to said pet. Specifically, for a dog weighing less than about 10 kg, or a cat, the 0.5% Dihydroquercetin composition is used mixing 1 package per meal in one meal a day for a dog or up to about 5 kg in weight, or a cat, and 1 package per meal in two meals a day for a dog between about 5 kg and about 10 kg in weight.

In another specific embodiment, for a dog weighing more than about 10 kg, the 1% by weight Dihydroquercetin composition is used mixing 1 package per meal in one meal a day if the dog is between about 10 kg in weight and about 50 lbs in weight and 1 package 10 per meal in two meals a day if the dog is over 50 lbs in weight. These methods are particularly useful one or more dogs have been exposed to parvo virus, as anti-oxidation, gut flora, and immune function are immediately enhanced when the dog needs this most.

In yet another embodiment of the invention, the health and nutritional composition is provided as a tablet formulated to be chewable by a pet. The Dihydroquercetin may be about 5 percent by weight or about 10 percent by weight, with the lower amount to be used for the smaller dogs and cats. The Dihydroquercetin and Larch Arabanogalactan may be present in an amount ranging from about 250 mg to about 2000 mg more specifically about 500 mg to about 1000 mg and most specifically about 1000 mg.

The chewable tablets may also be used in methods of enhancing antioxidation, gut flora and immune stimulation of a pet. For a dog weighing less than about 10 kg or a cat, the 5% Dihydroquercetin composition is used, and a dog weighing more than about 10 kg, the 10% Dihydroquercetin composition is used.

The chewable tablet, in another embodiment, may also include vitamin A, as beta-carotene; vitamin C, as ascorbic acid; vitamin D, as cholecalciferol, vitamin E, as tocopherol, thiamine, riboflavin, niacin, vitamin B-6 as pyridoxine hydrocholoride, pantothenic acid, vitamin B-12, biotin, and folic acid. The tocopherol may specifically be mixed tocopherols, dl-tocopherol acetate, and natural alpha tocopherol. In a highly specific embodiment, the vitamin A, as beta-carotene is present in an amount of about 1500 IU; the vitamin C, as ascorbic acid is present in an amount of about 20 mg; the vitamin D, as cholecalciferol is present in an amount of about 150 IU, the vitamin E, as tocopherol is present in an amount of about 15 IU; the thiamine is present in an amount of 4 mg; the riboflavin is present in an amount of about 650 mcg, the niacin is present in an amount of about 4 mg; the vitamin B-6 as pyridoxine hydrocholoride is present in an amount of about 360 mcg; the pantothenic acid is present in an amount of about 2 mg; the vitamin B-12 is present in an amount of about 8 mcg; the biotin is present in an amount of about 100 mcg; and folic acid is present in an amount of about 80 mcg.

In a related embodiment, the chewable tablets, either with or without the additional vitamins, are employed in methods of enhancing antioxidation, gut flora and immune stimulation of a pet by the administration of the chewable tablet as follows. In a dog weighing less than about 10 kg, or a cat, the 5% Dihydroquercetin composition is employed and is administered as 1 tablet to a pet if said pet is a cat or a dog weighing less than about 10 lbs or 2 tablets to said dog if said dog weighs between about 10 lbs and about 10 kg. In a dog weighing over about 10 kg, the 10% Dihydroquercetin composition is employed and is administered as follows: 1 tablet so a dog weighing between about 10 kg and about 20 kg, 2 tablets to a dog weighing between about 20 kg and about 45 kg, and 3 tablets to a dog weighing over 45 kg.

### EXAMPLE 1

One dog in a household of eight dogs developed and died of parvo virus. The other seven dogs were of mixed breeds including a Shitzu terrier, a Basset hound mix, two Coonhound mixes, a Cocker Spaniel, a small terrier and a young wolf hybrid ranging in weight from 8 to 50 lbs. These dogs were immediately treated with a health and nutritional composition consisting of 30 mg of Dihydroquercetin, 500 mg of Siberian or Dahurian Larch Arabinogalactan, and 70 mg of vitamin C, formulated in a single gel capsule. The following day and for four days thereafter, each dog received two gel capsules consisting of 30 mg of Dihydroquercetin, 500 mg of Siberian or Dahurian Larch Arabinogalactan, and 70 mg of vitamin C twice a day, for a total dose of 120 mg Dihydroquercetin, 2000 mg Siberian or Dahur Larch Arabinogalactan, and 280 mg of vitamin C per day. None of the seven (7) dogs treated with the composition developed signs or symptoms of parvo virus infection.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

1. An animal health and nutrition composition effective to enhance anti-oxidation, gut flora and immune stimulation in animals, the composition comprising:
a dihydroquercetin isolated from a Siberian or Dahurian larch; and
a Siberian or Dahurian larch arabinogalactan.

2. The animal health and nutrition composition of claim 1, wherein said Siberian or Dahurian larch arabinogalactan has a molecular weight between about 9.5 kDa and about 37 kDa.

3. The animal health and nutrition composition of claim 1, wherein said composition is in a forms of a tablet, chewable tablet, capsule, resin, oil, liquid, liposome, powder, or suppository.

4. The animal health and nutrition composition of claim 1, further comprising a pharmaceutically acceptable carrier.

5. The animal health and nutrition composition of claim 1, wherein said composition is incorporated into a bulk feed or a feed supplement.

6. The animal health and nutrition composition of claim 1, wherein said composition comprises 30 mg of said dihydroquercetin, 500 mg of said Siberian or Dahurian larch arabinogalactan, and 70 mg of a vitamin C.

7. The animal health and nutrition composition of claim 6, wherein said dihydroquercetin, said arabinogalactan and said vitamin C are formulated in a single gel capsule.

8. The animal health and nutrition composition of claim 1, wherein an amount of said dihydroquercetin is in range of 3%-30% by weight.
